(19) United States
(12) Patent Application Publication
(10) Pub. No.: US 2025/0312035 A1
(43) Pub. Date: Oct. 9, 2025

(54) SURGICAL FIXATION DEVICES, AND RELATED DELIVERY SYSTEMS AND METHODS

(71) Applicant: INTUITIVE SURGICAL OPERATIONS, INC., Sunnyvale, CA (US)

(72) Inventor: Christopher Julian, Los Gatos, CA (US)

(73) Assignee: INTUITIVE SURGICAL OPERATIONS, INC., Sunnyvale, CA (US)

(21) Appl. No.: 19/086,832

(22) Filed: Mar. 21, 2025

Related U.S. Application Data
(60) Provisional application No. 63/574,547, filed on Apr. 4, 2024.

(51) Int. Cl. A61B 17/064; A61B 17/10; A61B 17/34

(52) U.S. Cl. CPC A61B 17/0644; A61B 17/105; A61B 17/3468; A61B 2017/0647; A61B 2017/0649

(57) ABSTRACT

A system for delivering surgical fixation devices to a target region in a body includes an instrument comprising a shaft and a tissue puncturing member moveable in reciprocation relative to the shaft. The system further includes a plurality of surgical fixation devices positioned in series along at least part of a length of the shaft and configured to be deployed from the shaft without needing to remove and reinsert the instrument.

### CROSS-REFERENCE TO RELATED APPLICATIONS

This application claims priority to U.S. Provisional Application No. 63/574,547, filed Apr. 4, 2024, which is hereby incorporated by reference in its entirety.

### TECHNICAL FIELD

Aspects of the present disclosure relate to surgical fixation devices for fixation of material in a body, and related delivery systems and methods for use in minimally invasive procedures. In particular, aspects of the present disclosure relate to surgical fixation devices and systems for delivering surgical fixation devices for securing a reinforcement structure in a target site or region of a patient.

### INTRODUCTION

Surgical fixation devices have a variety of uses to attach to and/or secure objects in medical procedures, including to secure tissue and/or bone together, and/or to secure material to tissue and/or bone. Regarding the latter, some procedures involve using surgical fixation devices to attach reinforcement structures to bone and/or tissue to bridge, repair, and/or reinforce a defect. One such procedure includes hernia repair. A hernia is a development of a weakened area (which can include a gap) in the connective tissue of a patient's abdominal wall such that an organ or other tissue can bulge through. Various types of hernias exist corresponding generally to the specific location in the body.

To surgically repair hernias, a reinforcement structure, such as a surgical mesh, can be fixed within the fascia of the body to bridge and reinforce the weakened area in the abdominal wall and prevent tissue and/or organs, for example, the intestine, from pushing back into the weakened area after repair. Surgical fixation devices are used to secure the reinforcement structure in place and such fixation devices can take a number of forms, including, but not limited to, sutures, clips, anchors, tacks, screws and/or adhesive devices. Moreover, hernia repair procedures can be carried out either using open surgery or minimally invasive procedures, such as laparoscopic, percutaneous and/or robotic-assisted surgeries. The choice of fixation device and technique used may depend on the type of procedure as some fixation devices, such as traditional suturing, may be more difficult to use in a minimally invasive procedure due to space constraints.

Medical systems that operate at least in part with computer-assisted control and robotic technology (sometimes referred to as “telesurgical systems”, “robotic-assisted” or “robotic surgical systems”), such as those employed for minimally invasive medical procedures are used to perform a variety of minimally invasive medical procedures. The da Vinci® Surgical Systems commercialized by Intuitive Surgical, Inc. are examples of such computer-assisted medical systems.

Various challenges arise when performing minimally invasive medical procedures involving the application of surgical fixation devices, such as to attach a reinforcement structure in a hernia repair procedure. For example, spatial constraints and the trajectory of delivery of fixation devices cause challenges. Moreover, the location of nearby organs and/or other body tissue and structures can pose challenges in ensuring surgical fixation devices do not potentially impinge or damage the same. In the context of teleoperated, computer-assisted surgery, the ability to mimic conventional manual suturing techniques employed in an open surgical procedure can be particularly challenging and not practical. Further, the overall time of a procedure is a consideration and minimizing the same can be challenging if delivering and deploying fixation devices is relatively complex. Additionally, recovery and healing can pose challenges, which may be exacerbated in a region of the body, such as the abdomen in hernia repair procedures, which is subject to relatively large and frequent post-operative motion.

There exists a need for surgical fixation devices and systems for delivering surgical fixation devices, for example to attach reinforcement structures, that are both relatively easy to manufacture, deliver, and deploy independently of specific deployment trajectories or angles, thereby limiting the need for complex instrument articulations within regions of the body with space constraints. A need also exists for surgical fixation devices and systems for delivering surgical fixation devices that can deliver multiple fixation devices at a time so as to promote efficiency of a procedure. A need further exists for surgical fixation devices that minimize post-operative discomfort and pain. SUMMARY

Implementations of the present disclosure may solve one or more of the above-mentioned problems and/or may demonstrate one or more of the above-mentioned desirable features and address one or more of the above-mentioned needs. Other features and/or advantages may become apparent from the description that follows.

In accordance with at least one aspect, the present disclosure contemplates, a system for delivering surgical fixation devices to a target region in a body comprises an instrument comprising a shaft having a proximal end portion, a distal end portion, and a longitudinal axis, and a tissue puncturing member in the shaft and comprising a sharp distal end, the tissue puncturing member moveable in reciprocation relative to the shaft. The system further comprises a plurality of surgical fixation devices positioned in series along at least part of a length of the shaft, wherein each surgical fixation device comprises an elongate post having a proximal end portion and a distal end portion, a head at the proximal end portion of the elongate post, and one or more deployable members at the distal end portion of the elongate post, wherein the one or more deployable members are deployable from a collapsed configuration extending along the elongate post to an expanded configuration extending outwardly from the elongate post.

In at least another aspect, the present disclosure contemplates a system for delivering surgical fixation devices to a target region in a body comprises an instrument comprising a shaft configured to be inserted in the body; a suturing needle advanceable through tissue in the body; and a plurality of surgical fixation devices carried by the instrument and positioned in series along the shaft of the instrument. Each surgical fixation device of the plurality of surgical fixation devices comprises an elongate element having a proximal end portion, a distal end portion, and a curved shape between the proximal end portion and distal end portion, a planar support body having a first surface and a second surface opposite the first surface, and a needle docking element configured to receive a portion of the suturing needle. The proximal end portion of the elongate element is coupled to the first surface of the planar support body, and the distal end portion of the elongate element comprises a tissue anchoring structure comprising surface features on an exterior surface of the elongate element.

In yet an additional aspect, the present disclosure contemplates a system for delivering surgical fixation devices to a target region in a body comprises an instrument comprising a shaft having a proximal end portion, a distal end portion, and a longitudinal axis, and a cradle comprising a first end portion pivotably coupled to the distal end portion of the shaft. The cradle further comprises a closed end portion opposite the first end portion, a closed lateral portion, and an open lateral portion. The system further comprises a plurality of surgical fixation devices positioned in the shaft in series along the longitudinal axis of the shaft. Each surgical fixation device comprise a coil, and a sharpened tip at an end of the coil. The surgical fixation devices are moveable distally relative to the shaft to advance a distal-most helical coil through the open lateral portion in the cradle and out of the instrument.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description serve to explain certain principles and operation. In the drawings,

FIG. 1 is a schematic view of a system for delivering surgical fixation devices;

FIG. 2 is a schematic view of the system of FIG. 1 delivering surgical fixation devices to a target region in a body;

FIG. 3 is perspective view of a surgical fixation device in accordance with the present disclosure in a collapsed configuration;

FIG. 4 is a perspective view of the surgical fixation device of FIG. 3 in an expanded configuration;

FIG. 5 is a side perspective view of a system for delivering the surgical fixation devices of FIG. 3;

FIGS. 6A-6C are enlarged, perspective views of a distal end portion of an instrument of the system of FIG. 5, progressively illustrating delivery of the surgical fixation devices of FIG. 3 from a shaft of the instrument;

FIG. 7 is a side perspective view of the shaft of the instrument of the system of FIG. 5;

FIG. 8 is a side perspective view of a fixation device advancement member of the instrument of the system of FIG. 5;

FIG. 9 is a perspective view of a tissue puncturing member of the instrument of the system of FIG. 5;

FIGS. 10A-10E progressively illustrate the system of FIG. 5 during delivery of the surgical fixation devices of FIG. 3 for fixation of a reinforcement structure in a body;

FIG. 11 is perspective view of another surgical fixation device in accordance with the present disclosure;

FIG. 12 is perspective view of another surgical fixation device in accordance with the present disclosure;

FIG. 13 is a perspective view of an instrument for delivering the surgical fixation devices of FIG. 11;

FIG. 14 is a perspective view of a suturing needle and surgical fixation device of the system of FIG. 13;

FIG. 15 illustrates the suturing needle of FIG. 14 during delivery of the surgical fixation devices of FIG. 11 for fixation of a reinforcement structure in a body;

FIG. 16 is perspective view of another surgical fixation device in accordance with the present disclosure;

FIG. 17 is a partial, perspective view of a system for delivering the surgical fixation devices of FIG. 16;

FIG. 18 is perspective view of another surgical fixation device in accordance with the present disclosure;

FIG. 19 is a partial, perspective view of a system for delivering the surgical fixation devices of FIG. 18;

FIG. 20 is a partial, perspective view of another system for delivering surgical fixation devices;

FIG. 21 is an enlarged, perspective view of a distal end portion of an instrument of the system of FIG. 20;

FIG. 22 illustrates the system of FIG. 20 during delivery of a surgical fixation device for fixation of a reinforcement structure in a body; and

FIG. 23 is a perspective schematic view of a manipulator system according to an aspect of the present disclosure.

### DETAILED DESCRIPTION

The present disclosure contemplates various surgical fixation devices, such as fixation devices for fixation of a reinforcement structure in a patient's body. The present disclosure contemplates, for example, a variety of reinforcement structures, having various configurations and made of various materials, including, but not limited to patches, mesh structures (such, as, e.g., a biologic mesh (e.g., braided or woven) or a synthetic mesh made of a biocompatible material, such as metal for example), lattice structures, and/or plates (e.g., plates with holes). Related systems and methods for delivering such surgical fixation devices for securing the reinforcement structure also are disclosed. Implementations of the present disclosure can, for example, be used in minimally invasive procedures (e.g., laparoscopic and/or percutaneous), including, for example, both handheld systems and computer-assisted, teleoperated minimally invasive surgical systems (e.g., in conjunction with or as medical instruments connected to manipulator arms of telesurgical systems). Contemplated delivery systems can be manipulated independently of specific deployment trajectories or angles, thereby limiting the need for complex instrument articulations to deliver surgical fixation devices, such as within the tight abdominal spaces associated with various hernia repair procedures, including, for example, ventral hernia repair procedures.

Implementations of various contemplated delivery systems also allow for sequential deployment of surgical fixation devices at the target region, for example, sequential deployment of the surgical fixation devices at the target region without removal of the instrument from the body. Various implementations may also allow for successive, multi-fire operation of a delivery instrument to sequentially deploy surgical fixation devices one after another at the target region, thereby efficiently affixing a material, such as a reinforcement structure, to tissue at the target region. Simple and efficient surgical fixation device deployment may, for example, aid in reducing both the time and patient discomfort associated with a surgical procedure.

Further, various of the surgical fixation devices contemplated by the present disclosure may have low-profile design geometries and/or provide compliancy to further minimize post-operative discomfort and pain associated with the fixation devices and promote healing of the repaired target region.

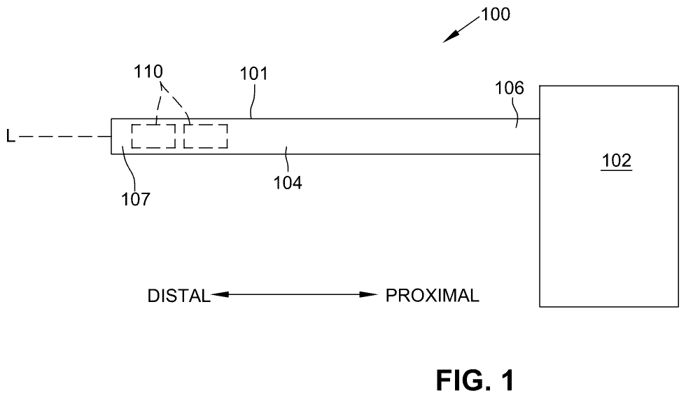

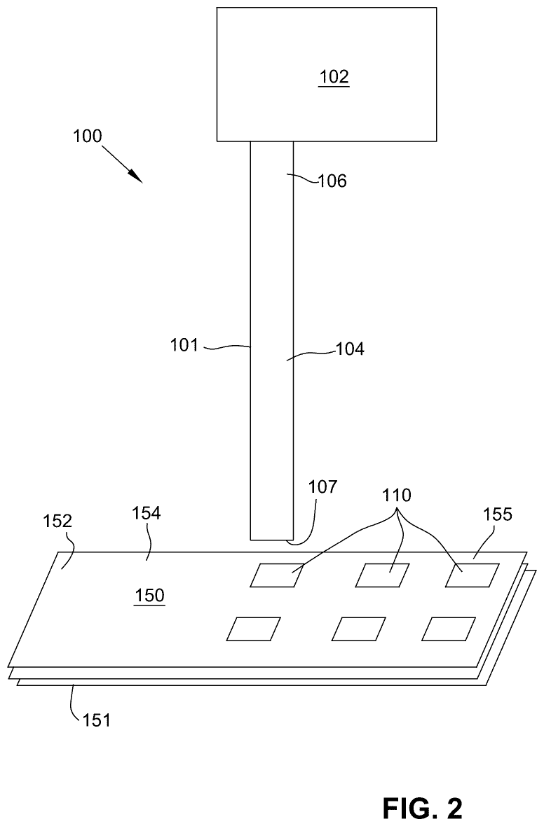

Referring now to FIGS. 1 and 2, schematic side views (with FIG. 1 shown to view an interior of a portion of the instrument) of one implementation of a system 100 for delivering surgical fixation devices 110 to a target region 150 (such as a target region 150 within a body of a patient) are shown. The system 100 includes an instrument 101 (such as, for example, a medical instrument) and a plurality of surgical fixation devices 110 carried by the instrument 101 for fixation into tissue 152 at the target region 150.

The instrument 101 includes a shaft 104 configured to be inserted in the body. The shaft 104 has a proximal end portion 106, a distal end portion 107, and a longitudinal axis L. The directions “proximal” and “distal” are used herein to define the directions as shown in FIG. 1, with distal generally being in a direction further along a kinematic arm or closest to the worksite in the intended operational use of the instrument 101, and proximal being along the direction further from the worksite. While aspects of the present disclosure are discussed in the context of minimally invasive instruments, such as, for example, instruments used during the repair of ventral hernias, implementations of the present disclosure are not so limited, and the principles disclosed herein can be applied to instruments used in open procedures or percutaneous procedures that use surgical fixation devices to attach objects. In addition, aspects of the disclosure can have non-surgical applications, such as in other remotely-actuatable instruments for inspection and other industrial uses, general robotic uses, manipulation of non-tissue work pieces, etc.

The instrument 101 further includes a transmission mechanism 102 located proximal a distal end portion of the shaft 104, such as at the proximal end portion 106 of the shaft 104. In an implementation, the transmission mechanism 102 is configured to interface with a manipulating system controlled in part through teleoperated, computer-assistance and robotic technology, such as, but not limited to, manipulating systems discussed below in connection with FIG. 23. Alternatively, the transmission mechanism 102 is configured to be operated manually such as for a manual, laparoscopic instrument, which can have a handle or other arrangement configured to be manipulated directly by a user.

The instrument shaft 104 is configured to carry a plurality of surgical fixation devices 110 for delivery of the surgical fixation devices 110 via the distal end portion 107 (e.g., distal end opening) of the shaft 104. As would be understood by those of ordinary skill in the art, delivery of the surgical fixation devices 110 can be controlled by manipulation of the transmission mechanism 102, either manually or through drives of a manipulating system (e.g., the manipulating system shown in FIG. 23). The transmission mechanism 102 includes various mechanical and/or electromechanical devices that transmit motion, energy, and/or signals, e.g., from the manipulating system, or from inputs at the transmission mechanism 102 operable by a user, to the instrument 101.

The transmission mechanism 102 is configured to operably couple to and receive drive inputs, such as from a manipulator system of a teleoperable, computer-assisted medical system that operates at least in part with robotic technology. One such non-limiting example of a computer-assisted medical system is illustrated in the schematic diagram of FIG. 23, which will be discussed in more detail below. However, as discussed above, the scope of the present disclosure contemplates the instrument 101 can be configured to be manually actuated, with the proximally located, transmission mechanism 102 having inputs that are configured to be manually actuated. Further contemplated are instruments that have both manually actuated inputs and inputs configured to be driven by drive outputs of a manipulator system.

As illustrated in FIG. 2, the instrument 101 may be used to sequentially deliver surgical fixation devices 110 to attach one object to another during a medical procedure. By way of a non-limiting example, the instrument 101 can be used in a hernia repair procedure, such as, for example, a ventral hernia repair, to secure a reinforcement structure 154 in place over the tissue 152 being repaired. The delivery of the surgical fixation devices 110 can be accomplished without removal and reinsertion of the instrument 101 through a body wall or other opening and/or incision of the patient.

For example, utilizing the system 100 to perform a ventral hernia repair procedure, the instrument 101 can be manipulated to sequentially place surgical fixation devices 110 at various locations along the reinforcement structure 154 (e.g., a mesh patch), to secure and fix the reinforcement structure 154 within fascia tissue 151 in an abdominal wall (i.e., to bridge and reinforce a weakened area in the abdominal wall) within the very limited space (i.e., tight abdominal space) afforded the surgeon during a ventral hernia procedure. In various embodiments, for example, the instrument 101 can be manipulated to sequentially place and fix a plurality of surgical fixation devices 110 at various positions of the reinforcement structure 154. In some implementations, the positions are around a periphery 155 of the reinforcement structure 154, other locations, including areas interior to the periphery 155 of the reinforcement structure 154, can also be used.

The design of the surgical fixation devices 110 can provide for one or more of various considerations. For example, the design can facilitate ease of delivery and deployment. Further, a relatively low-profile design geometry can be used such that when the surgical fixation devices are fixed within the tissue 152 at the target region 150 they are configured to minimize contact with surrounding tissue (e.g., the surrounding tissue of the abdominal wall), thereby also minimizing patient discomfort and pain that may be caused by irritations associated with the surgical fixation devices 110 (e.g., associated with chafing caused by the fixation devices 110). The surgical fixation devices 110 can be made of a bioabsorbable material, such as a bioabsorbable polymer, such that the surgical fixation devices 110 are configured to degrade and be absorbed naturally by the body at predictable rates as would be understood by those of ordinary skill in the art. The surgical fixation devices 110 can also be compliant or flexible so as to allow for post-operative movement of the body without undue impingement by the surgical fixation devices.

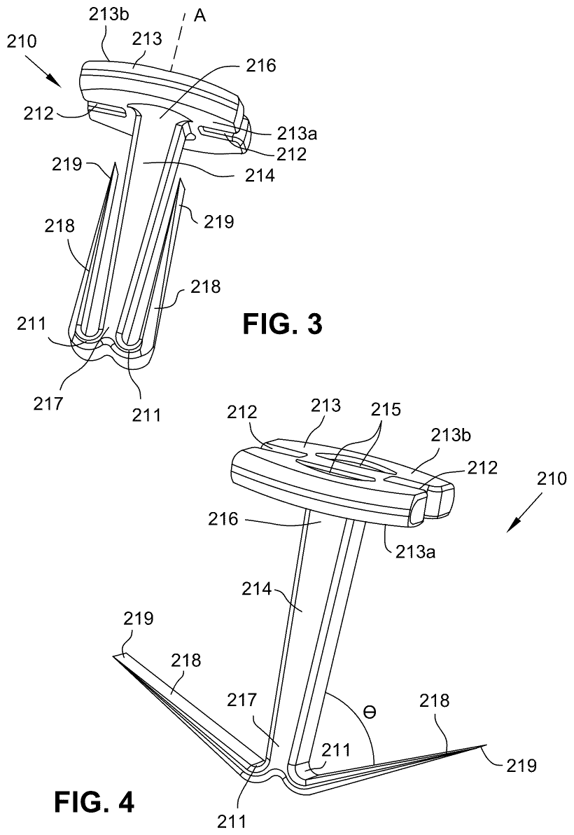

Referring now to FIGS. 3-10E, an implementation of a system for delivering surgical fixation devices to a target region in a body is illustrated and includes surgical fixation devices and an instrument for delivering and deploying multiple surgical fixation devices.

With reference to FIGS. 3 and 4, a surgical fixation device 210 in accordance with an implementation of the present disclosure. The surgical fixation device 210 includes an elongate post 214 extending along a longitudinal axis A and having a proximal end portion 216 and a distal end portion 217. The elongate post 214 is tapered along a proximal-to-distal direction, from the proximal end portion 216 to the distal end portion 217. Each surgical fixation device 210 also comprises a head 213 projecting laterally (e.g., radially) from the proximal end portion 216 of the elongate post 214. The head 213 has a first surface 213a facing toward the elongate post 214 and a second surface 213b, opposite the first surface 213a, facing away from the elongate post 214. In this manner, the elongate post 214 extends perpendicularly from the first surface 213a of the head 213 and the first surface 213a can have a relatively flat planar profile. The second surface 213b also may have a relatively flat planar profile (as illustrated), although a slightly rounded, convex surface profile is also contemplated. In either case, the second surface 213b can provide a surface profile that is substantially atraumatic if it were to come into contact with tissue.

As shown in FIG. 4, the head 213 also includes a pair of through holes 215 extending through the first and second surfaces 213a and 213b and positioned generally on opposite sides of the post 214. The through holes 215 can have a generally semicircular shape with the flat side of each through hole 215 facing radially inwardly and the curved side of each through hole 215 facing radially outwardly. However, this shape is non-limiting, and a variety of shapes can be used. As will be understood from the description below, the shape may be chosen based on the cross-sectional shape of portions of a delivery tool designed to be received in the through holes 215. The head 213 can further have a slots 212 which also are on opposite sides of the post 214, positioned diametrically opposite to each other and respectively spaced 90 degrees around the post 214 from the through holes 215. The slots 212 open to the periphery of the head 213.

The surgical fixation device 210 also comprises one or more deployable arm members 218, two deployable members 218 being shown in FIGS. 3 and 4, at the distal end portion 217 of the elongate post 214. The deployable members 218 are deployable from a collapsed configuration, in which the deployable members 218 extend along the elongate post 214 (see FIG. 3), to an expanded configuration, in which the deployable members 218 extend outwardly from the elongate post 214 (see FIG. 4). As shown, in the collapsed configuration, each of the deployable members 218 folds at the distal end portion 217 of the elongate post 214 and extends back toward the proximal end portion 216 of the elongate post 214, such that a free end portion 219 of each deployable member 218 extends toward the first surface 213a of the head 213 of the surgical fixation device 210. For example, in the collapsed configuration, each of the deployable members 218 can extend relatively parallel to the longitudinal axis A of the elongate post 214.

In the expanded configuration, the deployable members 218 can at least partially unfold away from the post 214 and expand outwardly to form an angle θ of 90 degrees or less (e.g., to form an angle θ ranging from an acute angle to a 90 degree angle) relative to the elongate post 214. The connection of the deployable members 218 to the post 214 can be via a living hinge 211 to allow for substantially elastic deformation between the collapsed, folded configuration of the deployable members 218 and the expanded, unfolded configuration of the deployable members 218. In both the folded, collapsed configuration and the expanded, deployed configuration of the deployable members 218, the distal end portion of the surgical fixation device 210 can be relatively blunt in profile so as to provide an atraumatic design should the surfaces come into contact with nearby tissue whether once implanted or during delivery.

In various implementations, the deployable members 218 are passively deployable from the collapsed configuration to the expanded configuration. For example, one or more portions of the deployable members 218 can be made of a super-elastic and/or shape-memory material, such that the deployable members 218 can self-expand from the collapsed configuration to the expanded configuration in the absence of a retaining force acting to keep the deployable members 218 in the folded, collapsed configuration. In this manner, as will be further discussed below, the deployable members 218 can be elastically deformed and held in the collapsed configuration and independently transition to the expanded configuration upon release of a force holding them in the collapsed configuration.

In another implementation, the fixation device 210 and deployable members can be composed of a polymer material (such as, for example, a bioabsorbable or biocompatible polymer) with the deployable members 218 molded in the expanded configuration. The molded deployable members 218 are resilient and/or deformable, such that the deployable members 218 are configured to be disposed into the collapsed configuration (e.g., folded or bent to abut or be disposed near the elongated post 214) and to be inserted and held in a delivery instrument 201 in the collapsed configuration. When a fixation device 210 is deployed from the instrument, the deployable members 218 can thus be biased to unfold (open or partially open) to the expanded configuration.

In various other implementations, the deployable members 218 are actively deployable from the collapsed configuration to the expanded configuration via, for example, a delivery instrument as will be discussed further below and/or through forces exerted during delivery by pulling the partially expanded deployable members 218 against tissue or a reinforcement structure. In some implementations, it is contemplated that the deployable members 218 are partially passively and partially actively deployable. For example, as will be described further below, a portion of a delivery instrument may be used to exert forces (e.g., push, press, move, translate) to assist in unfolding the deployable members to the expanded configuration, either from a partially expanded state due to being at least partially self-expandable or from a collapsed state for a deployable members that do not exhibit self-expansion properties.

The one or more deployable members 218 can have a variety of configurations and arrangements, such as the elongate arms that are positioned diametrically opposite each other as shown in FIGS. 3-10E. The arms can taper from where they connect to the post 214 (e.g., at the living hinge 211) to the free end portions 219. Further, the arms can have a relatively flat surface profile at the surface intended to come into contact with material in the deployed configuration. Those having ordinary skill in the art would appreciate that any number of deployable members could be used, and the positioning around the post 214 could be varied. Moreover, other shapes and/or geometries of the deployable members are contemplated as within the scope of the present disclosure, with the design considerations discussed herein.

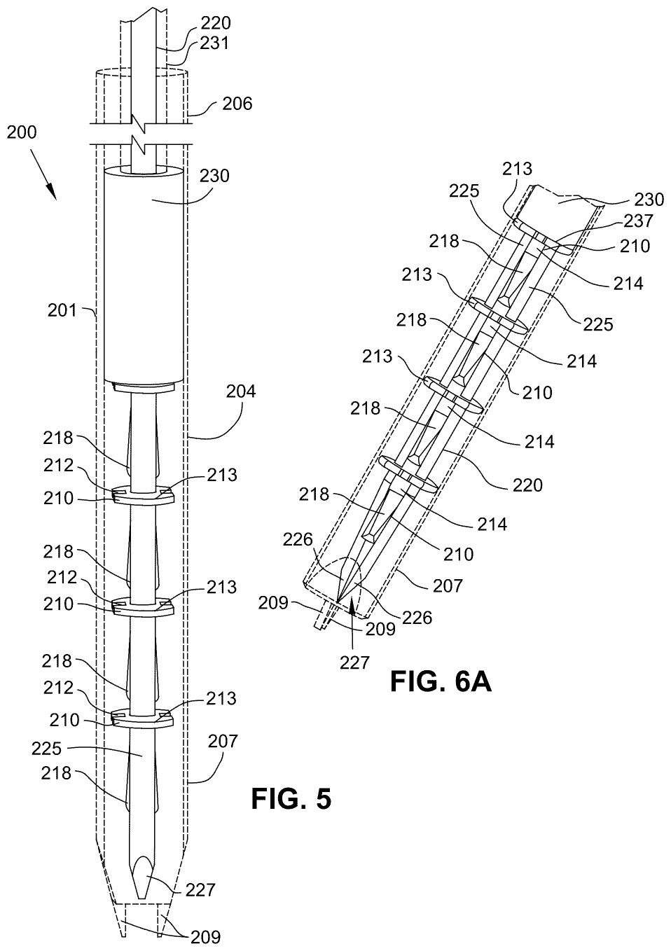

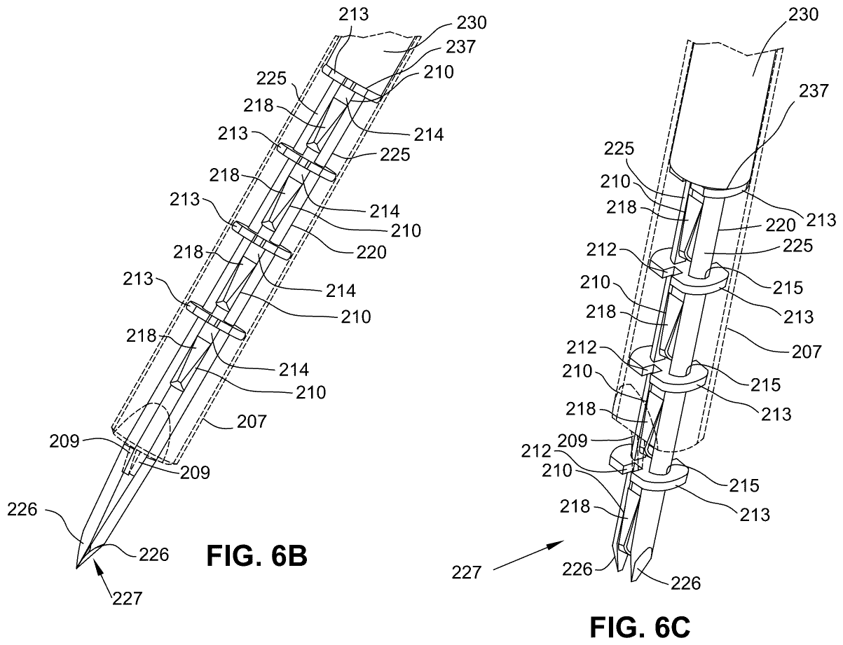

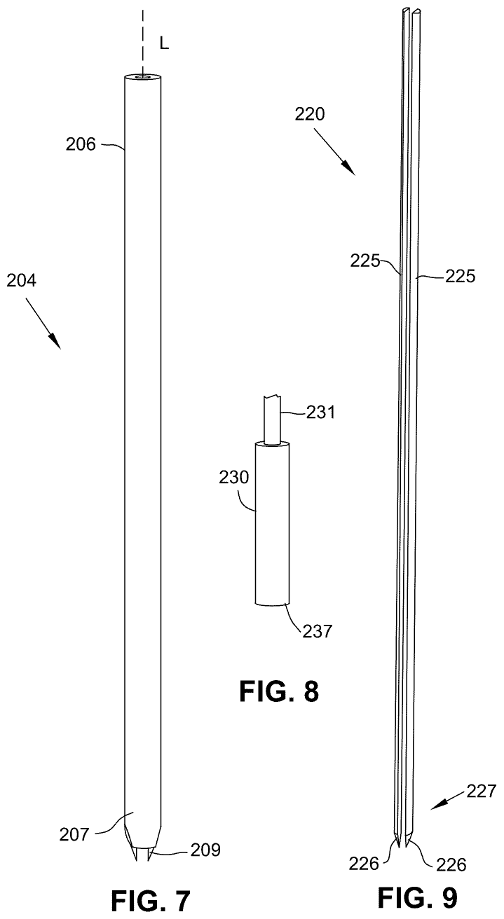

Referring now to FIGS. 5-7, a system 200 can include an instrument 201 configured for delivery and deployment of a plurality of the surgical fixation devices 210. The instrument 201 includes a hollow shaft 204 having a proximal end portion 206 and a distal end portion 207, and which is elongated along a longitudinal axis L (see FIG. 7). The instrument 201 also includes a tissue puncturing member 220 that extends through the lumen of the hollow shaft 204 and is moveable in reciprocal translation relative to the shaft 204 along the longitudinal axis L. The tissue puncturing member 220 comprises a sharp distal end 227 configured to puncture tissue. More specifically, the tissue puncturing member 220 comprises two elongated legs 225, with each leg terminating in a sharp distal tip 226, which together form the sharp distal end 227 of the tissue puncturing member 220. As shown in FIGS. 5 and 6C, the instrument 201 also includes a fixation device advancement member 230, which is configured to receive and move along an outer peripheral surface of the tissue puncturing member 220.

The fixation device advancement member 230 is moveable relative to the shaft 204 of the instrument 201 and the tissue puncturing member 220, and is actuated to move in a reciprocating manner (i.e., distally and proximally) independently of the tissue puncturing member 220. The fixation device advancement member 230 can be coupled to a drive shaft 231 (shown in dashed lines in FIG. 5) that in turn is driven by a proximally located transmission mechanism. For example, the shaft 231 can be hollow, thereby allowing the tissue puncturing member 220 to extend through and be moveable relative to the fixation device advancement member 230 and the shaft 231. In this way, the reciprocating motion of the tissue puncturing member 220 can occur relative to that of the fixation device advancement member 230. A distal end 237 of the fixation device advancement member 230 can be sized so as to contact the planar head 213 (i.e., abuts the second surface 213b of the head 213) of a proximal-most surgical fixation device 210 of the plurality of surgical fixation devices 210 held in series in the instrument shaft 204. The distal end of the fixation device advancement member 230 is sized to abut against the head 213 of the proximal-most fixation device 210.

As best illustrated perhaps in the views of FIGS. 5 and 6A-6C, in which the shaft 204 of the instrument 201 is shown in dash to illustrate the interior components of the system 200, in the assembled arrangement of the system 200 to deliver a plurality of surgical fixation devices 210, the surgical fixation devices 210 are all in the collapsed configuration and are in series and carried by the tissue puncturing member 220 such that they are held within the shaft 204 with the posts 214 oriented generally parallel to the longitudinal axis L of the shaft 204. As best shown in the enlarged views of FIGS. 6A-6C, the legs 225 of the tissue puncturing member 220 extend respectively through the pairs of through holes 215 in the head 213 of each of the surgical fixation devices 210. In this way, the elongate post 214 of each of the surgical fixation devices 210 is positioned between the two legs 225 of the tissue puncturing member 220 with the deployable members 218 positioned against the post 214 in the respective spaces between the legs 225.

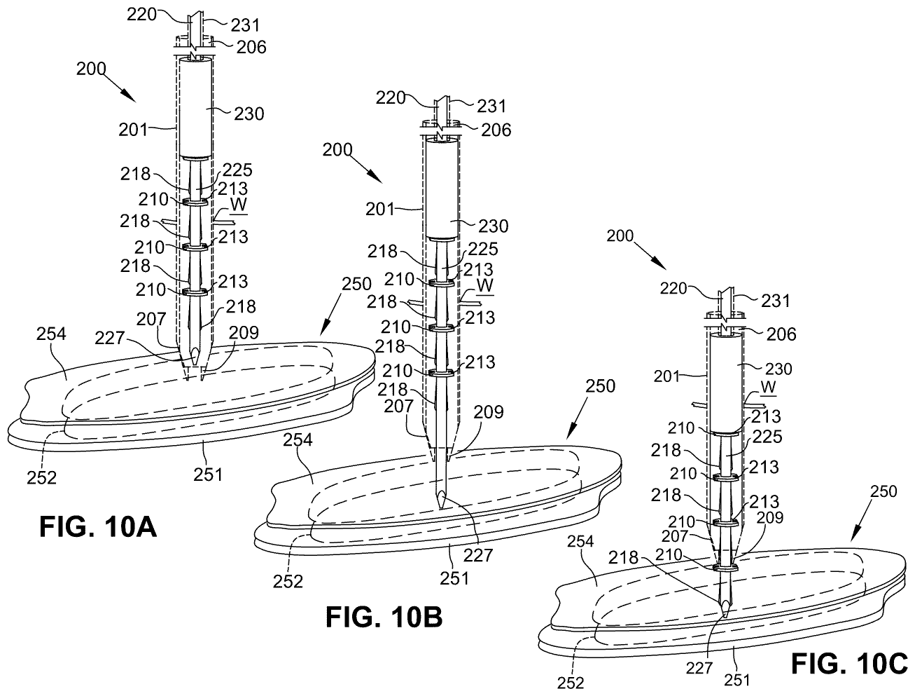

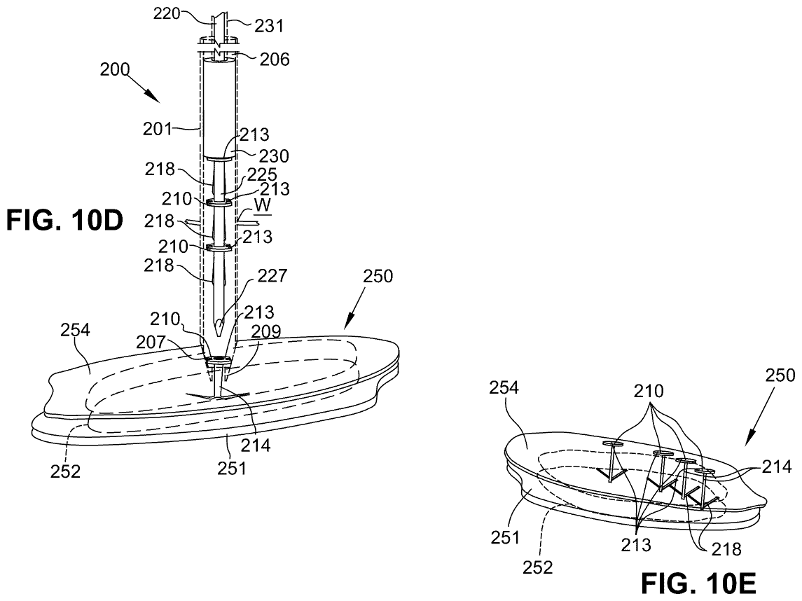

As further illustrated in the progressive views of FIGS. 6A-6C, and discussed further below with reference to FIGS. 10A-10E, to deliver the surgical fixation devices 210 (i.e., to a target region 250), the tissue puncturing member 220 is moved in a reciprocating manner in which it is configured to move relative to the shaft 204 distally past the distal end portion 207 of the shaft 204, for example, to drive the sharp distal end 227 (sharp distal tips 226) of the tissue puncturing member 220 into tissue 252 at the target region 250 to puncture the tissue 252. Actuation of the translational movement of the tissue puncturing member 220 can occur via a drive input at a proximally located transmission mechanism (e.g., transmission mechanism 102 not shown). Such input can occur either manually or via a manipulating system in a computer-assisted teleoperated system.

As shown in FIG. 6A, when inserted into the body and initially positioned at the target region 250, the tissue puncturing member 220 is retracted within the shaft 204 of the instrument with the surgical fixation devices 210 all in the collapsed configuration along the tissue puncturing member 220. The tissue puncturing member 220 is then translated distally, such that the sharp distal end 227 of the tissue puncturing member 220 is advanced past and out of the distal end portion 207, as illustrated in FIG. 6B. For example, the legs 225 of the tissue puncturing member 220 may move respectively through the pairs of through holes 215 in the head 213 of each of the surgical fixation devices 210 to advance the sharp distal end 227 without also advancing the surgical fixation devices 210 through which the tissue puncturing member 220 passes. In other words, the surgical fixation device 210 and tissue puncturing member 220 are slidable relative to each other to allow movement of the tissue puncturing member 220 to penetrate tissue and then allow a fixation device to be slid relative to the puncturing member 220 for deployment. In this manner, the sharp distal end 227 of the tissue puncturing member is used to puncture the tissue 252 at the target region 250, while the distal-most surgical fixation device 210 remains within the shaft 204.

As shown in FIG. 6C, the fixation device advancement member 230 may then be used to advance and deploy the distal-most fixation device 210 of the series of fixation devices 210 held in the instrument shaft 204. As the fixation device advancement member 230 moves distally, it pushes the series of fixation devices 210 distally as well due to the force exerted on the head 213 of the proximal-most fixation device 210, causing the fixation devices 210 to slide relative to the tissue puncturing member 220.

The fixation device advancement member 230 (shown in isolation in FIG. 8) is moveable in reciprocation along the outer peripheral surface of the tissue puncturing member 220 (shown in isolation in FIG. 9) along the longitudinal axis L of the instrument shaft 204, such that the fixation device advancement member 230 can move distally to advance the series of surgical fixation devices 210 toward the distal end portion 207 of the shaft 204. As noted above, and similar to the tissue puncturing member 220, actuation of the fixation device advancement member 230 can also occur via a proximally located transmission mechanism (e.g., transmission mechanism 102) and can occur in a timed fashion, but can move relative to and independently of movement of the tissue puncturing member 220. For example, the reciprocating motion of the fixation device advancement member 230 can occur through the use of a drive shaft 231, which can be in turn controlled by a proximally located transmission mechanism.

Actuation of the fixation device advancement member 230 advances the fixation device advancement member 230 in a distal direction so as to index the series of fixation devices 210 held in the instrument shaft 204 by a length of one fixation device 210, thereby pushing the distal-most fixation device 210 out of the distal end of the instrument shaft 204. The fixation device advancement member 230 then continues to move distally to allow for the deployment of the distal fixation device 210 into the target site. After deployment of the distal-most fixation device 210, the fixation device advancement member 230 can be retracted back into the instrument shaft 204, for example, by the amount of the continued advancement for the deployment of the previous fixation device 210, so as to be in position to permit the next distal-most fixation device 210 in the series to be deployed. The tissue puncturing member 220 also is retracted back into the shaft 204 for deployment of the next surgical fixation device 210, allowing the instrument 201 to be safely moved to the next desired location for deployment of the surgical fixation device 210.

As best shown in FIG. 7, depicting an isolated view of the shaft 204 of the instrument 201, the shaft 204 can further include guide members 209 shaped as tapered prongs that extend distally from the distal end of the shaft 204. The guide members 209 can be shaped to be received by the slots 212 as the head 213 of a surgical fixation device 210 moves through the distal end opening of the shaft 204 for deployment. The guide members 209 can also be used to aid in deployment of the deployable members 218 by using distal ends of the guide members 209 to push against the deployable members 218 once the fixation device 210 has been pushed past the distal end of the instrument shaft 204 and off the tissue puncturing member 220. In another implementation, the shaft 204 of the instrument 201 does not include the guide members 209 (not shown), such that the annular edge of the distal end portion 207 of the shaft 204 can be used to aid in deployment of the deployable members 218. The annular edge of the distal end portion 207 of the shaft 204 is configured to push the deployable members 218 of the deployed fixation device 210 while pushing the reinforcement structure 354 at the target region.

With reference now to the progressive views of FIGS. 10A-10E, as discussed above, an implementation of using the system 200 to sequentially deliver surgical fixation devices 210 to a target region 250 in a body of a patient will now be described. For example, the system 200 can be used during a hernia repair procedure to secure a reinforcement structure 254, such as, for example, a surgical mesh, in place over repaired tissue 252 and into fascia tissue 251 at the target region 250, without removal and reinsertion of the instrument 201 through a body wall (or other incision or opening) of the patient (e.g., for a ventral hernia repair procedure, without removal and reinsertion through the abdomen of the patient).

As shown in FIG. 10A, once inserted into the body, for example, through an incision or port in the abdomen wall W of the patient, the instrument 201 is positioned relative to a target region 250 within the abdomen. For example, the shaft 204 of the instrument 201 can be positioned such that the distal end portion 207 of the shaft 204 is adjacent to a surgical reinforcement structure 254 (e.g., a surgical mesh) used to strengthen repaired tissue 252 at the target region 250, with the longitudinal axis L of the shaft 204 extending substantially perpendicular to the surgical reinforcement structure 254. Once at a desired position for deployment of a surgical fixation device 210 carried by the instrument 201, the tissue puncturing member 220 is moved distally along the longitudinal axis L of the shaft 204, such that the sharp distal end 227 (e.g., comprising the sharp distal tips 226 of the legs 225) of the tissue puncturing member 220 is driven out of the distal end portion 207 of the shaft 204 to puncture the reinforcement structure 254 and underlying tissues 252 and 251, as shown in FIG. 10B.

As shown in FIG. 10C, the series of surgical fixation devices 210 are then advanced distally along the legs 225 of tissue puncturing member 220 by application of a distally directed force exerted by the fixation device advancement member 230, until the distal-most surgical fixation device 210 of the series of surgical fixation devices 210 is moved distally past the distal end portion 207 of the shaft 204 and slid off the distal end of the tissue puncturing member 220.

As illustrated in FIG. 10D, the deployable members 218 of the now released distal-most surgical fixation device 210 are then deployed from the collapsed configuration to the expanded configuration to secure the surgical fixation device 210 within the fascia tissue 251 at the target region 250, thereby also affixing the surgical reinforcement structure 254 to the fascia tissue 251. As discussed above, in various implementations, the deployable members 218 are passively deployable from the collapsed configuration to the expanded configuration, such that the deployable members 218 self-expand from the collapsed configuration to the expanded configuration once the surgical fixation device 210 is moved distally past the distal end portion 207 of the shaft 204 (e.g., once the collapsed deployable members 218 are no longer constrained by the shaft 204). In other implementations, the deployable members 218 are actively deployed from the collapsed configuration to the expanded configuration, for example, by the instrument 201 exerting a force on the deployable members 218 to expand them to the open position, after the surgical fixation device 210 is moved distally past the distal end portion 207 of the shaft 204. In some implementations, the deployable members 218 may be partially deployed, for example via self-expansion, and then before the surgical fixation device 210 is fully removed from the tissue puncturing member 220, the shaft 204 can be moved proximally such that the deployable members 218 engage with the tissue 251 and the forces cause further expansion of the deployable members 218 and/or the guide members 209 can be used to aid in expanding the deployable members 218 by applying a force to open them as described above.

Once the distal-most surgical fixation device 210 is deployed, the instrument 201 is then repositioned relative to the target region 250 and the above procedure repeated to deliver additional surgical fixation devices 210 carried by the instrument 201. In this manner, the instrument 201 can be manipulated to sequentially place and fix a plurality of surgical fixation devices 210 around the reinforcement structure 254 (e.g., around a peripheral region), as illustrated in FIG. 10E. As discussed above, the instrument 201 can be manipulated to secure and fix the surgical fixation devices 210 at varying frequency and positions, including areas along a perimeter and/or in an interior region of the reinforcement structure 254.

The delivery systems 200 and related methods discussed above with reference to FIGS. 3-10E, including the instruments 201 and surgical fixation devices 210 utilized therein, are exemplary only, and the present disclosure contemplates various types and configurations of systems for sequentially delivering surgical fixation devices to a target region in a body of a patient, which utilize various types and configurations of instruments and surgical fixation devices, several of which will be further discussed below.

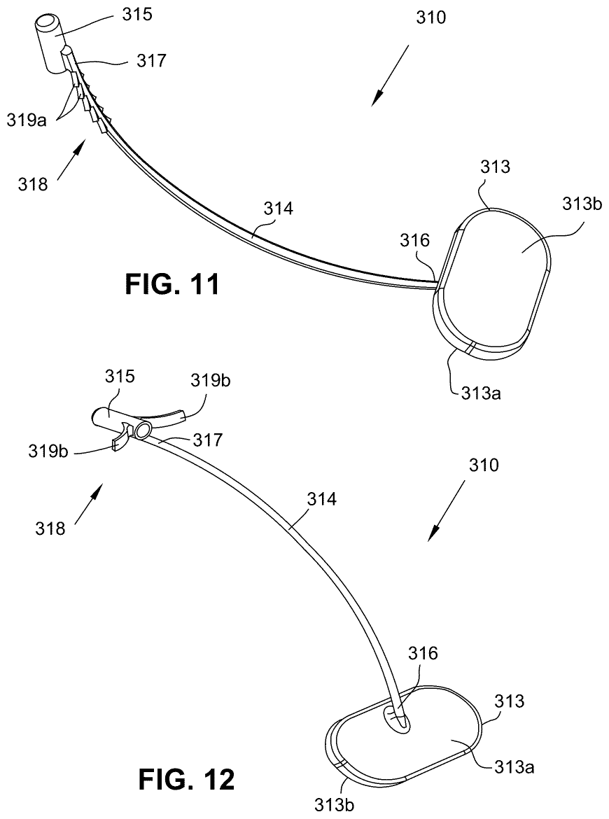

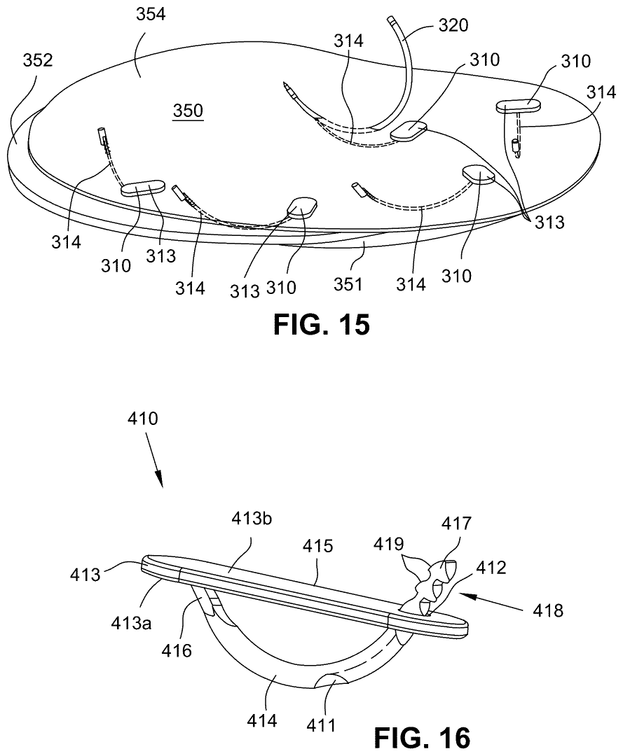

In various additional implementations, the present disclosure contemplates systems utilizing surgical fixation devices that are intended to mimic traditional sutures in certain aspects of their delivery and deployment. Referring now to FIGS. 11-15, surgical fixation devices 310 and a system 300 for delivery of a plurality of such fixation devices to a target region 350 (see FIG. 15) in a body are discussed.

With reference to FIGS. 11 and 12, a surgical fixation device 310 comprises an elongate element 314 with a laterally protruding head 313 at one end portion and a needle docking element 315 at an opposite end portion. The needle docking element 315 has a hollow tubular configuration sized to receive a portion of a suturing needle 320, allowing the sharp tip of the needle 320 to extend past the distal end of the needle docking element 315 (described further below with reference to FIG. 14). The head 313 has a first surface 313a and a second surface 313b opposite the first surface 313a. The surfaces 313a, 313b can be substantially planar and the head 313 can be relatively thin in the dimensions perpendicular to the surfaces 313a, 313b, thus having a pad-like configuration. In this manner, when the surgical fixation device 310 is delivered to the target region 350, as described further below, the head 313 of the fixation device 310 can provide a surface profile that is substantially atraumatic if it were to come into contact with tissue.

The elongate element 314 has a proximal end portion 316 that is coupled to the head 313 (e.g., at the first surface 313a of the planar support body of the head 313) and a curved shape extending from the proximal end portion 316 to a distal end portion 317 of the elongate element 314.

The distal end portion 317 of the elongate element 314 comprises a tissue anchoring structure 318, which includes surface features on an exterior surface of the elongate element 314 that are configured to be inserted into and embedded in tissue at the target region 350. For example, the surface features, as illustrated in FIG. 11, can be self-locking, barbed surface features 319a, which allow for adjustment of the elongate element 314 by controlling a depth of penetration of the distal end portion 317 in tissue. In another example, as illustrated in FIG. 12, the distal end portion 317 of the elongate element 314 includes self-locking, winged surface features 319b.

The surgical fixation device 310 can be made of a medical grade molded plastic, which may be bioabsorbable. The elongate element 314 can be provided with some degree of bendability and thus, with the tissue anchoring structure 318, the surgical fixation device 310 can act as a self-locking pledgit. In this manner, the surgical fixation device 310 can be compliant or flexible so as to allow for post-operative movement of the body without undue impingement by the surgical fixation devices 310.

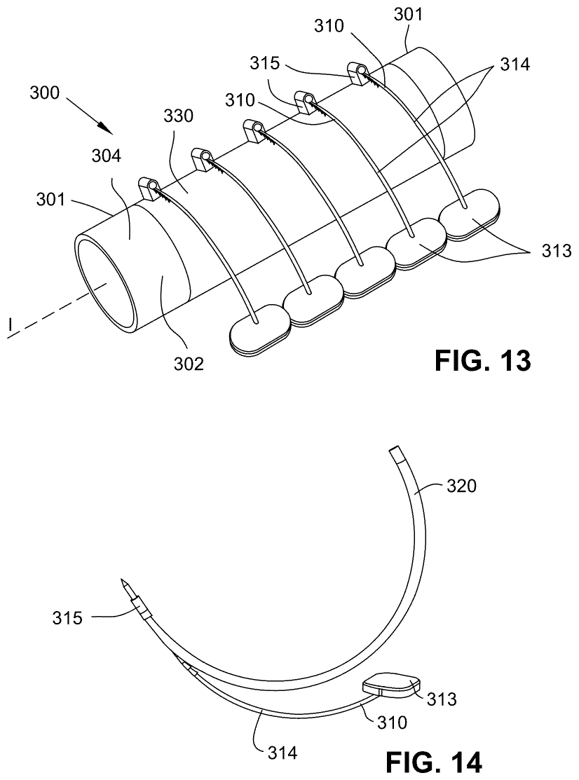

A system for delivery and deployment of a plurality of the surgical fixation devices 310 is illustrated in FIGS. 13-15. In one implementation, a system 300 for delivery of a plurality of surgical fixation devices 310 can utilize a traditional curved needle suturing instrument, such as a needle driver instrument (not shown), and a shaft 304 of another instrument 301, such as, a grasper instrument, to carry the fixation devices 310 (see FIG. 13 showing partial perspective view of shaft 304 and FIGS. 14 and 15 showing curved needle 320 of suturing instrument).

As illustrated in FIG. 13, in an assembled arrangement the surgical fixation devices 310 are positioned in series along the shaft 304 of the instrument 301 and are arranged generally perpendicular to the longitudinal axis I. For example, the needle docking element 315 of each surgical fixation device 310 can be clipped or engaged with a corresponding feature on an exterior surface 302 of the shaft 304, while the curved shape of the elongate element 314 of each surgical fixation device 310 is draped over the exterior surface 302 such that the head 313 of each surgical fixation device 310 hangs freely. In an alternative implementation, the system 300 can include a sleeve or cuff 330 that can carry the surgical fixation devices 310 and can be placed over the shaft 304 of the instrument 301 such that it at least partially surrounds the exterior surface 302 of the shaft 304 to carry the plurality of surgical fixation devices 310. In this implementation, the needle docking element 315 of each surgical fixation device 310 is clipped onto the sleeve 330 instead of directly to the exterior surface 302 of the shaft 304. Those of ordinary skill in the art would understand that the surgical fixation devices 310 may be positioned in series along the shaft 304 of the instrument 301 in various arrangements and while using various features and techniques, and the embodiments of FIGS. 11-15 are not intended to be limited to the exemplary arrangements and techniques disclosed herein.

As illustrated in FIGS. 14 and 15, to deliver the surgical fixation devices 310 to a target region 350, a suturing needle 320 of a suturing instrument (only the needle portion shown in FIG. 14) is inserted through the needle docking element 315 of one of the surgical fixation devices 310 of the plurality of surgical fixation devices 310 to remove the selected surgical fixation device 310 from the shaft 304. Then, similar to a suture, the suturing needle 320, the sharp tip of which protrudes distally past the needle docking element 315, may pass the elongate element 314 through a surgical reinforcement structure 354 (e.g., surgical mesh) and repaired tissue 352 and fascia tissue 351 at the target region 350. The head 313 attached to the elongate element 314 follows along until it abuts the reinforcement structure 354. Then the needle 320 can continue as in a traditional suturing technique back up into the tissue at a location spaced from the location of the head 313 to embed the surface features (e.g., 319a or 319b) of the tissue anchoring structure 318 at the distal end portion 317 of the elongate element 314 into the tissues 352, 351. Once the tissue anchoring structure 318 is embedded in the tissues 352, 351, the surgical fixation device 310 is fixed in place and the suturing needle 320 can then be retracted proximally from the needle docking element 315 of the surgical fixation device 310 to remove the suturing needle 320, while leaving the surgical fixation device 310 implanted at the target region 350.

In one implementation, the needle docking element 315 can have a surface profile that assists with retraction and removal of the needle 320. For example, the inner surface of the needle docking element 315 defining the hollow lumen that receives the needle 320 can be tapered in the distal direction and rotation of the needle 320 allows the inner surface of the needle docking element 315 to assist with the release and retraction of the needle 320 from the needle docking element 315.

In another implementation, prior to retraction of the needle 320 from the needle docking element 315, the needle docking element 315 can be squeezed (for example, using a grasper instrument) against the suturing needle 320, and the suturing needle 320 can be pulled to ratchet the barbs 315a or wings 315b against the reinforcement structure 354. The needle docking element 315 can then be released and the suturing needle 320 retracted from the needle docking element 315, leaving the implanted fixation device 310. The suturing needle 320 may then retrieve a subsequent surgical fixation device 310 and the process may be sequentially repeated until the surgical reinforcement device 354 is adequately attached and secured to the repaired tissue at the target region 350.

In various other implementations, the suturing needle 320 may instead be deployable using the instrument 301. For example, the instrument 301 may comprise a grasper type end effector that is configured manipulate the suturing needle 320 and a sleeve 330 carrying the series of surgical fixation devices 310 can be fitted onto the grasper instrument 301. In yet other alternative implementations, the suturing needle can be deployed using a laparoscopic, manual instrument for example in an open surgical procedure.

Referring now to FIGS. 16-19, various other implementations of surgical fixation devices and systems for delivery of a plurality of surgical fixation devices are illustrated. In these implementations, surgical fixation devices 410 have a configuration similar in some respects to the surgical fixation devices 310 with a curved elongated element 414 and an enlarged head to provide a pad-like structure that abuts a reinforcement structure in the deployed state of the surgical fixation device 410. A system 400 further includes an instrument 401 for carrying a plurality of the surgical fixation devices 410 and a suturing needle 420 for delivering the surgical fixation devices 410 and implanting them in tissue and relative to the reinforcement structure.

With reference to FIG. 16, similar to the embodiment of FIGS. 11-15, each surgical fixation device 410 comprises a head 413, an elongate element 414, and a needle docking element that is configured to receive a portion of the suturing needle 420. The head 413 has a first surface 413a and a second surface 413b opposite the first surface 413a. The surfaces 413a, 413b can be substantially planar and the head 413 can be relatively thin in the dimensions perpendicular to the surfaces 413a, 413b, thus having a pad-like configuration similar to that of FIGS. 11-15. In this manner, when the surgical fixation device 410 is delivered to a target region, the head 413 of the fixation device 410 also provides a surface profile that is substantially atraumatic if it were to come into contact with tissue.

The elongate element 414 has a proximal end portion 416 that is coupled to the first surface 413a of the head 413 and a curved shape extending from the proximal end portion 416 to a distal end portion 417 of the elongate element 414. In various embodiments, the proximal end portion 416 of the elongate element 414 is coupled to the first surface 413a of the head 413 toward one end of an elongated body 415 of the head 413 via a living hinge allowing the elongate element 414 to pivot relative to the head 413. The head 413 can include an engagement feature 412, such as an aperture as illustrated, through which the free, distal end portion 417 of the elongate element 414 can pass at an opposite end of the elongated body 415 of the head 413.

The distal end portion 417 of the elongate element 414 comprises an anchoring structure 418, which includes surface features 419 on an exterior surface of the elongate element 414 that are configured to be inserted into and pass through the aperture 412 in one direction and prevent the distal end portion 417 from disengaging from the aperture 412 once inserted. For example, the distal end portion 417 of the elongate element 414 includes barbed surface features 419.

While barbed surface features 419 and aperture 412 can provide for engagement and securing of the distal end portion 417 of the elongate element 414 to the head 413, other engagement features, such as snap-fit complementary features (e.g., a bulbous portion on the elongate element configured to snap-fit in a recess in the surface of the head), and the like can be used instead.

In various implementations, the needle docking element can include an aperture in the elongate element 414 with at least a portion of the elongate element 414 along the length distal to the opening being hollow to allow for a suturing needle (e.g., a curved suturing needle of a needle driver instrument) to advance through the opening in the elongate element 414 and pass through the hollow portion of the elongate element 414 such that the sharp distal tip of the needle extends past the distal end of the elongate element 414 to allow for delivery and deployment of the same in the target region, in a manner similar to the embodiment of FIGS. 11-15 described above and as is explained further below. Needle docking and delivery is described in more detail below.

When deployed at the target region, the surgical fixation device 410 functions similar to a clip, such that the head 413 of the surgical fixation device 410 forms a backing surface, with material to be captured being secured between the head 413 and the elongate element 414 of the surgical fixation device 410. The surgical fixation device 410 can be made of a medical grade molded plastic, which may be bioabsorbable, and the elongate element 414 can be provided with some degree of bendability. In this manner, the surgical fixation device 410 can be compliant or flexible so as to allow for post-operative movement of the body without undue impingement by the surgical fixation device 410.

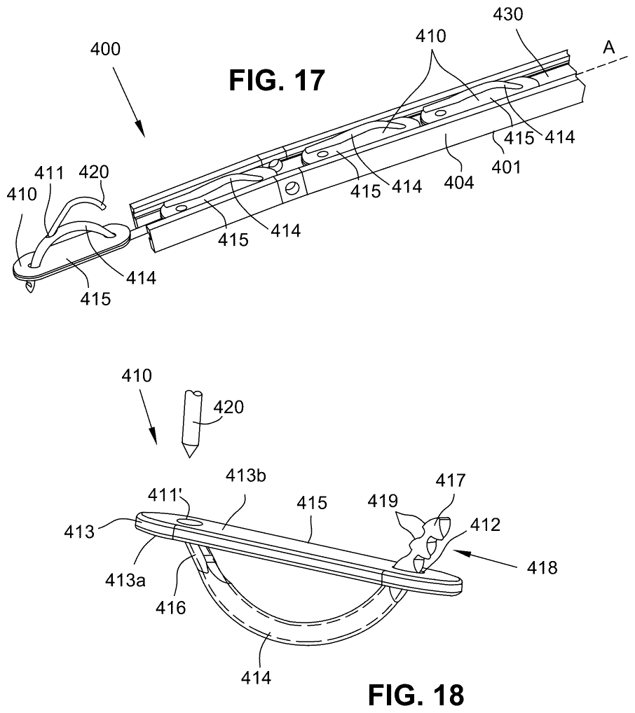

Referring now to FIG. 17, a system 400 for delivery of a plurality of the surgical fixation devices 410 is shown. The instrument 401 includes a shaft 404 that is elongated along a longitudinal axis A and is configured to be inserted in a body (e.g., through an abdomen of a patient for a ventral hernia repair procedure). As illustrated in FIG. 17, in an assembled arrangement of the system 400, the surgical fixation devices 410 are positioned in series along the shaft 404 of the instrument 401 and are arranged such that the elongate dimension of the fixation devices 410 are generally parallel to the longitudinal axis A of the shaft 404. In various embodiments, for example, the planar, elongated body 415 of each surgical fixation device 410 is positioned within a slot 430 in the shaft 404 of the instrument 401 and is configured to slide distally relative to the shaft 404 within the slot 430 for delivery to the target region.

A suturing needle 420 can be driven similar to a sewing machine in a reciprocating manner to pick up and deliver each surgical fixation device 410 in the series held in the instrument shaft 404 and once a surgical fixation device 410 is delivered, the needle 420 can be retracted back and pick up the next, distal-most surgical fixation device 410 in the series. In a remotely-controlled, robotic application, the drive system could be automated at the proximal transmission end of the instrument and coupled with a manipulator to control the driving; alternatively, the needle could be driven by a manual laparoscopic instrument.

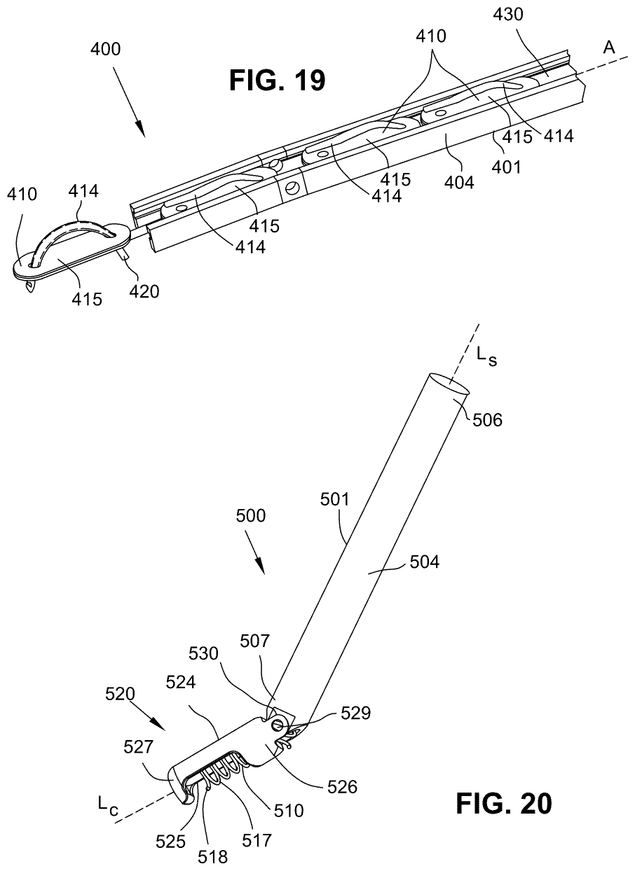

To deliver the surgical fixation devices 410 to a target region, a suturing needle 420 of the system 400 is advanced into the needle docking element of the distal-most surgical fixation device 410 of the plurality of surgical fixation devices 410 to slide the surgical fixation device 410 out of the slot 430 in the shaft 404. With reference to FIGS. 16 and 17, in one implementation the needle docking element can include an aperture 411 provided in a side wall of the elongate element 414, which, for example, can be located midway to distal of midway along a length of the elongate element 414, though such positioning is not limited. At least a portion of the elongate element 414 from the location of the aperture 411 and distally can be hollow to allow the suturing needle 420 to be inserted into the elongate element 414 for manipulation and deployment of the surgical fixation device 410, as further described below. In an alternative implementation, shown in FIGS. 18 and 19, the needle docking element can include an aperture 411′ (see FIG. 18) at a proximal end of the elongate element 414 and the length of the elongate element 414 can be hollow to allow the suturing needle 420 to advance through the elongate element 414 and carry the surgical fixation device 410 for deployment.

It is further contemplated that in either implementation, the elongate element 414 can comprise an opening toward a distal end that allows for the sharpened tip of the suturing needle 420 to advance out of the elongate element 414 for penetration of tissue and deployment of the surgical fixation device 410, in a manner similar to that described with respect to FIG. 15.

After docking the needle 420, similar to a suture, the suturing needle 420 may pass the free, distal end portion 417 of the elongate element 414 of the surgical fixation device 410 down through a surgical reinforcement structure and underlying tissues (e.g., repaired tissue and fascia tissue) at the target region. The head 413 attached to the elongate element 414 follows along until it abuts the reinforcement structure, for example in a manner similar to that described above with reference to FIG. 15. Then the needle 420 can continue as in a traditional suturing technique back up into the tissue to direct the free, distal end portion 417 of the elongate element 414 back toward and in alignment with the aperture 412 of the head 413. The head 413 can then be manipulated and pivoted so that the distal end portion 417 of the elongate element 414 passes through the aperture 412 and is secured relative to the head 413 via the barbed surface features 419 of the anchoring structure 418. For example, a separate grasper instrument may be used and/or the suturing needle may be used to manipulate the remainder of the surgical fixation device 410 to pass the elongate element 414 through the aperture 412. The suturing needle 420 can then be retracted back through the elongate element 414 and withdrawn from the aperture 411, 411′, while leaving the surgical fixation device 410 implanted at the target region.

In accordance with yet additional implementations, the present disclosure contemplates surgical fixation device delivery systems that utilize fixation devices in the form of helical coils, which can be compression springs. Referring to FIGS. 20-22, a system 500 for delivering surgical fixation devices 510 to a target region 550 (see FIG. 20) in a body is illustrated. The system 500 includes an instrument 501 and a plurality of surgical fixation devices 510 carried by the instrument 501. Each surgical fixation device 510 comprises a helical coil 517 with a sharpened tip 518 at distal end of the helical coil 517.

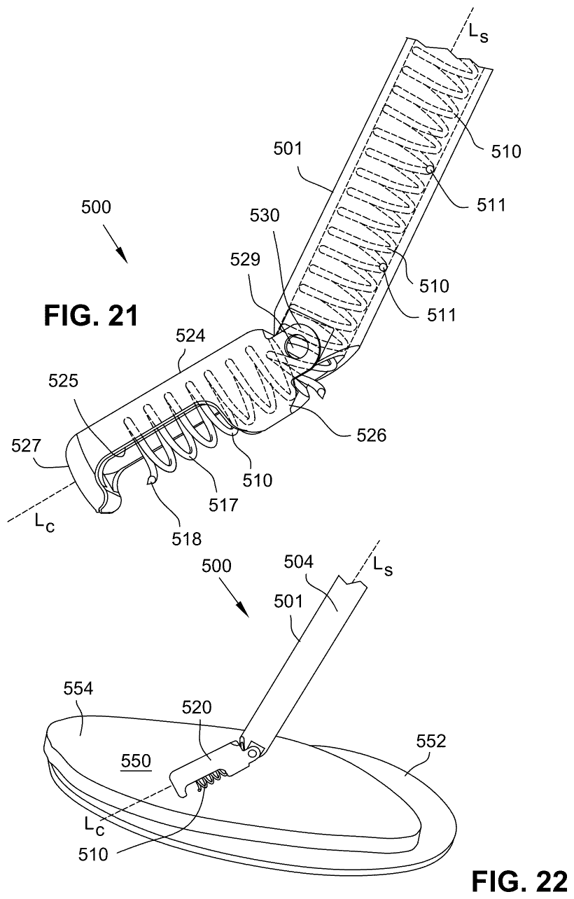

The instrument 501 includes a shaft 504 having a proximal end portion 506 and a distal end portion 507, and which is elongated along a longitudinal axis Ls (see FIG. 20). The instrument 501 also includes a cradle 520 that is elongated along a longitudinal axis L_{C} and comprises a first end portion 526 and a second, closed end portion 527 opposite the first end portion 526. The first end portion 526 is pivotably coupled to the distal end portion 507 of the shaft 504 to form a joint 529 between the shaft 504 and the cradle 520. The cradle 520 also includes a closed lateral portion 524 and an open lateral portion 525 (e.g., window, lateral aperture, or the like), such that the open lateral portion 525 forms an elongated opening in the cradle 520, which is configured to expose at least a portion of a distal-most surgical fixation device 510 of a series of surgical fixation devices 510 (see FIG. 21). The cradle 520 is pivotably coupled to the shaft 504, for example, via a hinge 530 or other similar pivot coupling, such that the cradle 520 can pivot and articulate about the hinge 530 to orient the longitudinal axis of the cradle L_{C} at various angles relative to the longitudinal axis L_{S} of the shaft 504.

As perhaps best illustrated in the enlarged view of FIG. 21, in which the surgical fixation devices 510 are shown in dash within the shaft 504 of the instrument 501, in the assembled arrangement of the system 500, the surgical fixation devices 510 are positioned in series within the shaft 504 along the longitudinal axis L_{S} of the shaft 504. In various embodiments, for example, the helical coils 517 are positioned in series, in an end-to-end arrangement, within the shaft 504, such that the coils 517 are moveable distally relative to the shaft 504 to advance a distal-most helical coil 517 of the series of helical coils 517 through the open lateral portion 525 in the cradle 520 of the instrument 501. In various embodiments, to aid with the distal advancement of the helical coils 517 within the shaft 504, a coupler 511 may be used to join adjacent ends of the helical coils 517 together. In this manner, as the distal-most helical coil 517 is advanced distally and out through the open lateral portion 525 of the cradle 520, the entire chain of helical coils 517 is moved distally.

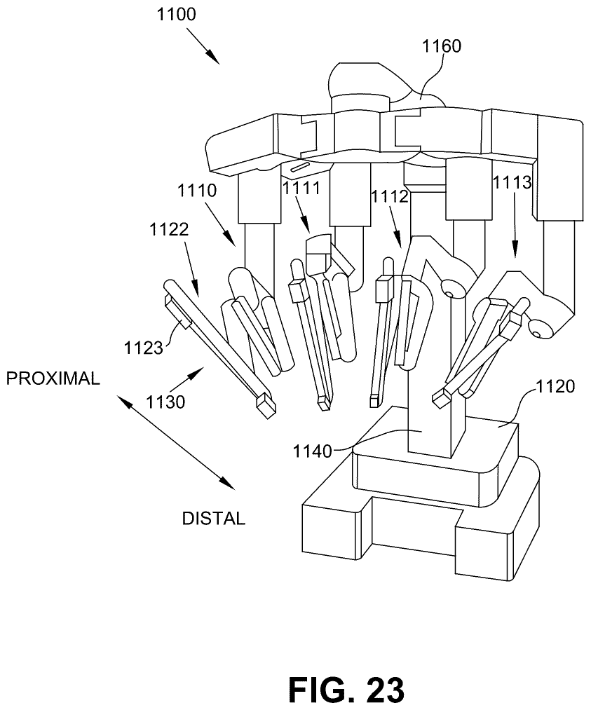

As illustrated in the view of FIG. 22, to deliver the surgical fixation devices 510 comprising the helical coils 517 to a target region 550, the closed end portion 527 of the cradle 520 is forced against a surface at the target region 550. For example, the closed end portion 527 of the cradle 520 is forced against a reinforcement structure 554 at the target region 550 such the cradle 520 of the instrument 501 can be pivoted relative to the shaft 504 of the instrument 501 (e.g., via the hinge 530 at the joint 529), to orient the opening of the cradle 520 generally parallel and abutting the surface of the reinforcement structure 554. Once oriented as desired to expose the sharpened tip 518 of the distal-most helical coil 517 to the surface of the reinforcement structure 554, the instrument 501 can be manipulated to drive the sharpened tip 518 of the distal-most helical coil 517 (which is exposed via the open lateral portion 525 of the cradle) through both the reinforcement structure 554 and an underlying tissue 552 (which can include a repaired tissue layer and a fascia layer as has been described herein). As would be understood by those of ordinary skill in the art, a penetration depth of the sharpened tip 518 of the distal-most helical coil 517 is a function of both a diameter of the distal-most helical coil 517 and a lateral force applied by the instrument 501 to the tissue 552 and can be adjusted accordingly.

For example, during delivery of the distal-most helical coil 517, the cradle 520 is configured to pivot relative to the shaft 504 of the instrument 501 until the open lateral portion 525 of the cradle 520 is positioned relatively flush against a surface of the target region 550 (against the reinforcement structure 554). The shaft 504 along with the cradle 520 of the instrument 501 are then rotated to insert (e.g., screw) the distal-most helical coil 517 into the tissue 552 at the target region 550, as the distal-most helical coil 517 is advanced through the open lateral portion 525 of the cradle 520 of the instrument 501. For sequential delivery of the helical coils 517 at the target region 550, as the distal-most helical coil 517 is advanced distally and out through the open lateral portion 525 of the cradle 520, the entire chain of helical coils 517 is moved distally, until the next distal-most helical coil 517 is positioned within the cradle 520 with its sharpened tip 518 exposed by the open lateral portion 525 of the cradle 520. In another implementation, the distal-most helical coil 517 is driven by rotation applied to the distal-most helical (e.g., force applied by the cradle 520) causing the chain of helical coils 517 to be pulled or moved distally for sequential delivery of the helical coils 517.

When each sequential helical coil 517 is deployed, the coupler 511 that joins the distal-most helical spring with the next distal-most helical spring (e.g., adjacent helical springs 517) is detached or decoupled. The detachment of the coupler 511 may occur by exertion of forces (e.g., mechanical connection coupler), by application of energy (e.g., electrolytical joint coupler) or any other suitable detachment techniques. In one implementation, the coils can be manufactured in series with weak points (e.g., snap-fit and/or corrugation) as the couplers joining adjacent coils so that manipulation of the coils during deployment can include breaking at the weak points. The coils can be made of biocompatible metal or plastic, and can be bioabsorbable.

In yet another implementation, it is contemplated the coils are separate from each other and driven through the shaft and cradle 524 by a separate element, such as, for example, a screw type drive mechanism. To assist with the coils not getting stuck as they are driven, the cradle 524 can be provided with threading on an interior surface that engages the distal-most coil, while the coil adjacent to that does not enter the cradle until subsequent driving of the series of coils after deployment of the distal-most coil.

As illustrated in FIG. 21, in an articulated orientation of the cradle 520 of the instrument 501 relative to the shaft 504 of the instrument 501, the surgical fixation devices 510 (e.g., the helical coils 517) are each resiliently bendable to traverse the joint 529 connecting the distal end portion 507 of the shaft 504 of the instrument 501 to the first end portion 526 of the cradle 520 of the instrument 501. In this manner, the instrument 501 only need be repositioned, and the process repeated, for sequential delivery of the surgical fixation devices 510 to secure the reinforcement structure 554 at the target region 550.

As discussed above, those of ordinary skill in the art will understand that the delivery systems 200, 300, 400, and 500, including the instruments 201, 301, 401, and 501 and the surgical fixation devices 210, 310, 410, and 510, discussed and illustrated with reference to FIGS. 3-22, are exemplary only and that systems and methods for delivery surgical fixation devices in accordance with the present disclosure can have various types and configurations of components, including and in addition to the instruments 201, 301, 401, and 501 and the surgical fixation devices 210, 310, 410, and 510, to sequentially deliver surgical fixation devices to target regions within tight surgical spaces, such as for example, to laparoscopically deliver surgical fixation devices within an abdomen of a patient during a ventral hernia repair procedure. Indeed, those of ordinary skill in the art would understand that a particular application (e.g., type of medical procedure), with a defined set of design considerations, may dictate the type and configuration of components used in the systems.

As discussed above, in accordance with various embodiments, force transmission systems of the present disclosure are configured for use in teleoperated, computer-assisted surgical systems employing robotic technology (sometimes referred to as robotic surgical systems). Referring now to FIG. 23, an embodiment of a manipulator system 1100 of a computer-assisted surgical system, to which surgical instruments are configured to be mounted for use, is shown. Such a surgical system may further include a user control system, such as a surgeon console (not shown) for receiving input from a user to control instruments coupled to the manipulator system 1100, as well as an auxiliary system, such as auxiliary systems associated with the da Vinci® systems noted above.

As shown in the embodiment of FIG. 23, the manipulator system 1100 includes a base 1120, a main column 1140, and a main boom 1160 connected to the main column 1140. Manipulator system 1100 also includes a plurality of manipulator arms 1110, 1111, 1112, 1113, which are each connected to the main boom 1160. Manipulator arms 1110, 1111, 1112, 1113 each include an instrument mount portion 1122 to which an instrument 1130 may be mounted, which is illustrated as being attached to the manipulator arm 1110. While the manipulator system 1100 of FIG. 23 is shown and described having a main boom 1160 to which the plurality of manipulator arms 1110, 1111, 1112, 1113 are coupled and supported thereby, in other embodiments, the plurality of manipulator arms 1110, 1111, 1112, 1113 can be coupled and supported by other structures, such as an operating table, a ceiling, wall, or floor of an operating room, etc.

Instrument mount portion 1122 comprises a drive output assembly 1123 (which may interface, for example, generally with the transmission mechanism 102 of the instrument 101 described above with reference to FIGS. 1 and 2), according to an embodiment. Although the embodiment of FIG. 23 shows an instrument 1130 attached to only manipulator arm 1110 for ease of viewing, an instrument may be attached to any and each of the manipulator arms 1110, 1111, 1112, 1113.

Other configurations of surgical systems, such as surgical systems configured for single-port surgery, are also contemplated.

Embodiments described herein may be used, for example, with remotely operated, computer-assisted systems (such, for example, teleoperated surgical systems) such as with various da Vinci® Surgical Systems, commercialized by Intuitive Surgical, Inc., of Sunnyvale, California.

The embodiments described herein are not limited to the surgical systems noted above, and various other teleoperated, computer-assisted surgical and manipulator system configurations may be used with the embodiments described herein. Further, although various embodiments described herein are discussed in connection with a manipulating system of a teleoperated surgical system, the present disclosure is not limited to use with a teleoperated surgical system. Various embodiments described herein can optionally be used in conjunction with hand-held, manual instruments.

As further discussed above, the embodiments described herein can be well suited for use in medical applications. Such procedures could be performed, for example, on human patients, animal patients, human cadavers, animal cadavers, and portions or human or animal anatomy. Some embodiments can also be suitable for use in, for example, for human or animal anatomy, training medical or non-medical personnel, and procedures on tissue removed from human or animal anatomies (without return to the human or animal anatomy). Even if suitable for use in such medical procedures, the embodiments can also be used for benchtop procedures on non-living material and forms that are not part of a human or animal anatomy. Moreover, some embodiments are also suitable for use in non-medical applications, such as industrial robotic uses

As used herein and in the claims, terms such as computer-assisted or teleoperable in referencing manipulator systems, or the like should therefore be understood to refer broadly to any system comprising one or more controllable kinematic structures (“manipulators”) that are movable and controllable at least in part through the aid of an electronic controller (with or without human inputs). Such systems can occasionally be referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) can facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems can be implemented in a variety of contexts to perform a variety of procedures, both medical and non-medical. Thus, although some examples described in greater detail herein can be focused on a medical context, the devices and principles described herein are also applicable to other contexts, such as industrial manipulator systems.

Illustrative aspects and combinations of contemplated surgical fixation devices and related delivery systems are provided below. Implementations of the surgical fixation devices and systems described herein may include any one or more, and any combination of, the clauses described below:
Clause 1. A system for delivering surgical fixation devices to a target region in a body, the system comprising:
   an instrument comprising:
   a shaft having a proximal end portion, a distal end portion, and a longitudinal axis, and
   a tissue puncturing member in the shaft and comprising a sharp distal end, the tissue puncturing member reciprocably moveable along the longitudinal axis of the shaft; and
   a plurality of surgical fixation devices carried by the tissue puncturing member and positioned in series parallel to the longitudinal axis of the shaft, wherein each surgical fixation device comprises:
   an elongate post having a proximal end portion and a distal end portion,
   a head projecting from the proximal end portion of the elongate post, and
   one or more deployable members at the distal end portion of the elongate post,
   wherein the one or more deployable members are deployable from a collapsed configuration extending along the elongate post to an expanded configuration extending outwardly from the elongate post.
Clause 2. The system of clause 1, wherein the tissue puncturing member extends through the head of each of the surgical fixation devices.
Clause 3. The system of clause 2, wherein the head of each of the surgical fixation devices comprises through holes positioned diametrically opposite to one another on the head and the tissue puncturing member comprises two legs respectively extending through the through holes.
Clause 4. The system of clause 3, wherein the elongate post of each of the surgical fixation devices is positioned between the two legs of the tissue puncturing member.
Clause 5. The system of clause 1, wherein the tissue puncturing member is configured to move distally past a distal end portion of the shaft.
Clause 6. The system of clause 5, wherein the surgical fixation devices are slidable along the tissue puncturing member.
Clause 7. The system of clause 1, wherein the one or more deployable members of each surgical fixation device are configured to passively deploy from the collapsed configuration to the expanded configuration.
Clause 8. The system of clause 1, wherein the one or more deployable members of each surgical fixation device are configured to be deployed from the collapsed configuration to the expanded configuration.
Clause 9. The system of clause 1, wherein in the collapsed configuration, the one or more deployable members fold at the distal end portion of the elongate post back toward the proximal end portion of the elongate post with a free end portion of each deployable member extending toward the head.
Clause 10. The system of clause 1, wherein in the expanded configuration, the one or more deployable members form an angle ranging from an acute angle to a 90-degree angle relative to the elongate post.
Clause 11. The system of clause 1, wherein the elongate post is tapered along a proximal-to-distal direction.
Clause 12. The system of clause 1, wherein the head is substantially planar with a first surface facing toward the elongate post and a second surface opposite the first surface, facing away from the elongate post, and wherein the elongate post extends from the first surface of the head.
Clause 13. A system for delivering surgical fixation devices to a target region in a body, the system comprising:
   an instrument comprising a shaft configured to be inserted in the body;
a suturing needle advanceable through tissue in the body; and
   a plurality of surgical fixation devices carried by the instrument and positioned in series along the shaft of the instrument, wherein each surgical fixation device of the plurality of surgical fixation devices comprises:
   an elongate element having a proximal end portion, a distal end portion, and a curved shape between the proximal end portion and distal end portion,
   a planar support body having a first surface and a second surface opposite the first surface, and
   a needle docking element configured to receive a portion of the suturing needle,
   wherein the proximal end portion of the elongate element is coupled to the first surface of the planar support body, and
   wherein the distal end portion of the elongate element comprises a tissue anchoring structure comprising surface features on an exterior surface of the elongate element.
Clause 14. The system of clause 13, wherein the suturing needle is deployable from the shaft of the instrument.
Clause 15. The system of clause 13, wherein the suturing needle is distinct from the instrument.
Clause 16. The system of clause 13, further comprising a sleeve carrying the plurality of surgical fixation devices and mounted on the shaft of the instrument.
Clause 17. A system for delivering surgical fixation devices to a target region in a body, the system comprising:
   an instrument comprising:
   a shaft having a proximal end portion, a distal end portion, and a longitudinal axis, and
   a cradle comprising a first end portion pivotably coupled to the distal end portion of the shaft, the cradle further comprising a closed end portion opposite the first end portion, a closed lateral portion, and an open lateral portion; and
   a plurality of surgical fixation devices positioned in the shaft in series along the longitudinal axis of the shaft, each surgical fixation device comprising:
   a helical coil, and
   a sharpened tip at an end of the helical coil,
   wherein the surgical fixation devices are moveable distally relative to the shaft to advance a distal-most helical coil through the open lateral portion in the cradle and out of the instrument.
Clause 18. The system of clause 17, wherein rotation of the shaft of the instrument is configured to screw the distal-most helical coil into tissue at the target region as the distal-most helical coil is advanced through the open lateral portion of the cradle of the instrument.
Clause 19. The system of clause 17, wherein, in an articulated orientation of the cradle of the instrument relative to the shaft of the instrument, the surgical fixation devices are each bendable to traverse a joint connecting the distal end portion of the shaft of the instrument to the first end portion of the cradle of the instrument.
Clause 20. The system of clause 17, wherein the cradle of the instrument is configured to pivot relative to the shaft of the instrument such that the open lateral portion of the cradle is positioned against a surface of the target region.
Clause 21. A surgical fixation device for fixation of material in a body, the surgical fixation device comprising:
   an elongate post having a proximal end portion, a distal end portion, and a longitudinal axis;
   a head projecting from the proximal end portion of the elongate post; and
   one or more deployable members at the distal end portion of the elongate post,
   wherein the one or more deployable members are deployable from a collapsed configuration extending along the elongate post to an expanded configuration extending outwardly from the elongate post,
   wherein the distal end portion of the elongate post terminates in a blunt surface profile in the collapsed configuration of the one or more deployable members.
Clause 22. The surgical fixation device of clause 21, wherein the elongate post is tapered in a proximal-to-distal direction.
Clause 23. The surgical fixation device of clause 21, wherein the head is substantially planar with a first surface facing toward the elongate post and a second surface opposite the first surface, facing away from the elongate post, and wherein the elongate post extends from the first surface of the head.
Clause 24. The surgical fixation device of clause 21, wherein the head comprises slots positioned diametrically opposite to one another on the head.
Clause 25. The surgical fixation device of clause 21, wherein the one or more deployable members are passively deployable from the collapsed configuration to the expanded configuration.
Clause 26. The surgical fixation device of clause 21, wherein the one or more deployable members are actively deployable from the collapsed configuration to the expanded configuration.
Clause 27. The surgical fixation device of clause 21, wherein, in the collapsed configuration, the one or more deployable members fold at the distal end portion of the elongate post back toward the proximal end portion of the elongate post with a free end portion of each deployable member extending toward the head.
Clause 28. The surgical fixation device of clause 21, wherein, in the expanded configuration, the one or more deployable members for an angle ranging from an acute angle to a 90-degree angle relative to the elongate post.
Clause 29. The surgical fixation device of clause 21, wherein the one or more deployable members are made of a super-elastic and/or shape memory material.
Clause 30. The surgical fixation device of clause 21, wherein the one or more deployable members are connected to the distal end portion of the elongate post via a living hinge.
Clause 31. The surgical fixation device of clause 21, wherein the surgical fixation device is made of a bioabsorbable material.
Clause 32. The surgical fixation device of clause 21, wherein the one or more deployable members comprise a plurality of arms positioned at differing circumferential locations about the elongate post.
Clause 33. The surgical fixation device of clause 32, wherein the plurality of arms comprises two arms positioned diametrically opposite one another.
Clause 34. A surgical fixation device for fixation of material in a body, the surgical fixation device comprising:
   an elongate element having a proximal end portion, a distal end portion, and a curved shape between the proximal end portion and the distal end portion; and
   a planar support body having a first surface and a second surface opposite the first surface,
   wherein the proximal end portion of the elongate element is coupled to the first surface of the planar support body, and
   wherein the distal end portion of the elongate element comprises a tissue anchoring structure comprising surface features on an exterior surface of the elongate element configured to be inserted into and embedded in tissue in the body.
Clause 35. The surgical fixation device of clause 34, wherein the surface features comprise barbs.
Clause 36. The surgical fixation device of clause 34, wherein the surface features comprise wings.
Clause 37. The surgical fixation device of clause 34, wherein the surgical fixation device is made of a bioabsorbable material.
Clause 38. The surgical fixation device of clause 34, further comprising a needle docking element configured to releasably receive a portion of a suturing needle.
Clause 39. The surgical fixation device of clause 38, wherein the needle docking element comprises a tube at the distal end portion of the elongate element.
Clause 40. The surgical fixation device of clause 34, wherein the planar support body comprises an aperture configured to receive the distal end portion of the elongate element.
Clause 41. The surgical fixation device of clause 34, wherein the proximal end portion of the elongate element is coupled to the first surface of the planar support body via a living hinge.

This description and the accompanying drawings that illustrate embodiments should not be taken as limiting. Various mechanical, compositional, structural, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the figures and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

What is claimed is:

1. A system for delivering surgical fixation devices to a target region in a body, the system comprising:
an instrument comprising:
a shaft having a proximal end portion, a distal end portion, and a longitudinal axis, and
a tissue puncturing member in the shaft and comprising a sharp distal end, the tissue puncturing member moveable in reciprocation relative to the shaft; and
a plurality of surgical fixation devices positioned in series along at least part of a length of the shaft, wherein each surgical fixation device comprises:
an elongate post having a proximal end portion and a distal end portion,
a head at the proximal end portion of the elongate post, and
one or more deployable members at the distal end portion of the elongate post,
wherein the one or more deployable members are deployable from a collapsed configuration extending along the elongate post to an expanded configuration extending outwardly from the elongate post.

2. The system of claim 1, wherein the tissue puncturing member extends through the head of each of the surgical fixation devices.

3. The system of claim 2, wherein the head of each of the surgical fixation devices comprises through holes positioned diametrically opposite to one another on the head and the tissue puncturing member comprises two legs respectively extending through the through holes.

4. The system of claim 3, wherein the elongate post of each of the surgical fixation devices is positioned between the two legs of the tissue puncturing member.

5. The system of claim 1, wherein the tissue puncturing member is configured to move distally past a distal end portion of the shaft.

6. The system of claim 5, wherein the surgical fixation devices are slidable along the tissue puncturing member.

7. The system of claim 1, wherein the one or more deployable members of each surgical fixation device are configured to passively transition from the collapsed configuration to the expanded configuration.

8. The system of claim 1, wherein the one or more deployable members of each surgical fixation device are configured to be at deployed from the collapsed configuration to the expanded configuration.

9. The system of claim 1, wherein in the collapsed configuration, the one or more deployable members fold at the distal end portion of the elongate post back toward the proximal end portion of the elongate post with a free end portion of each deployable member extending toward the head.

10. The system of claim 1, wherein in the expanded configuration, the one or more deployable members form an angle ranging from an acute angle to a 90-degree angle relative to the elongate post.

11. The system of claim 1, wherein the elongate post is tapered along a proximal-to-distal direction.

12. The system of claim 1, wherein the head is substantially planar with a first surface facing toward the elongate post and a second surface opposite the first surface, facing away from the elongate post, and wherein the elongate post extends from the first surface of the head.

13. A system for delivering surgical fixation devices to a target region in a body, the system comprising:
an instrument comprising a shaft configured to be inserted in the body;
a suturing needle advanceable through tissue in the body; and
a plurality of surgical fixation devices carried by the instrument and positioned in series along the shaft of the instrument, wherein each surgical fixation device of the plurality of surgical fixation devices comprises:
an elongate element having a proximal end portion, a distal end portion, and a curved shape between the proximal end portion and distal end portion,
a planar support body having a first surface and a second surface opposite the first surface, and
a needle docking element configured to receive a portion of the suturing needle,
wherein the proximal end portion of the elongate element is coupled to the first surface of the planar support body, and
wherein the distal end portion of the elongate element comprises a tissue anchoring structure comprising surface features on an exterior surface of the elongate element.

14. The system of claim 13, wherein the suturing needle is deployable from the shaft of the instrument.

15. The system of claim 13, wherein the suturing needle is distinct from the instrument.

16. The system of claim 13, further comprising a sleeve carrying the plurality of surgical fixation devices and mounted on the shaft of the instrument.

17. A system for delivering surgical fixation devices to a target region in a body, the system comprising:
an instrument comprising:
a shaft having a proximal end portion, a distal end portion, and a longitudinal axis, and
a cradle comprising a first end portion pivotably coupled to the distal end portion of the shaft, the cradle further comprising a closed end portion opposite the first end portion, a closed lateral portion, and an open lateral portion; and
a plurality of surgical fixation devices positioned in the shaft in series along the longitudinal axis of the shaft, each surgical fixation device comprising:
a coil, and
a sharpened tip at an end of the coil,
wherein the surgical fixation devices are moveable distally relative to the shaft to advance a distal-most helical coil through the open lateral portion in the cradle and out of the instrument.

18. The system of claim 17, wherein rotation of the shaft of the instrument is configured to screw the distal-most helical coil into tissue at the target region as the distal-most helical coil is advanced through the open lateral portion of the cradle of the instrument.

19. The system of claim 17, wherein, in an articulated orientation of the cradle of the instrument relative to the shaft of the instrument, the surgical fixation devices are each bendable to traverse a joint connecting the distal end portion of the shaft of the instrument to the first end portion of the cradle of the instrument.

20. The system of claim 17, wherein the cradle of the instrument is configured to pivot relative to the shaft of the instrument such that the open lateral portion of the cradle is positioned against a surface of the target region.
